(19) United States
(12) Patent Application Publication
(10) Pub. No.: US 2024/0182830 A1
(43) Pub. Date: Jun. 6, 2024

(54) MICROWELLS FOR CELLULAR SPHEROID ASSEMBLY

(71) Applicant: 4DCELL, Montreuil (FR)

(72) Inventors: Ana Rita DA SILVA RODRIGUES RIBEIRO, Montreuil (FR); Maël LE BERRE, Montreuil (FR)

(73) Assignee: 4DCELL, Montreuil (FR)

(21) Appl. No.: 18/074,286

(22) Filed: Dec. 2, 2022

(51) Int. Cl. C12M 1/32; A61L 29/08; C12M 1/00

(52) U.S. Cl. CPC C12M 23/12; A61L 29/085; C12M 23/20

(57) ABSTRACT

A cell culture device comprising at least one microwell comprising a top portion including a top opening for introducing cells, a bottom portion for cultivating said cells and a side wall joining said top portion to said bottom portion for guiding cells from said top opening to said bottom portion, characterized in that said top portion and said side wall are integral and made of a first material non-adhesive to cells and said bottom portion is made of a second material adhesive to cells.

### TECHNICAL FIELD

The present invention concerns a cell culture device comprising at least one microwell having a top portion including a top opening for introducing cells, a bottom portion for cultivating the cells and a side wall joining the top portion to the bottom portion for guiding cells from said top opening to said bottom portion.

### BACKGROUND

Preliminary phases in the development of new drugs usually use animal models (typically including mice, rats, guinea pigs, hamsters, gerbils, and rabbits) and/or so called two-dimensional (2D) cell cultures that comprise monolayers of cells.

However, only 10% of the molecules that initially enter phase I of clinical trials are finally brought to market at the end of the study [cf. Hay, M., Thomas, D. W., Craighead, J. L., Economides, C., & Rosenthal, J. (2014)—Clinical development success rates for investigational drugs. Nature biotechnology, 32(1), 40-51]. This is mainly because neither experiments on animal models nor tests on monolayers of cells can predict with sufficient precision the impact that the treatment with the drug will have on the human body.

Although experiments on animals are still necessary to observe phenomena on the scale of an entire organism, the physiology of animals is too different from that of humans in order to be able to transpose the observations made on the animals with sufficient certainty to the human body. Moreover, experiments on animals are expensive and raise ethical questions.

2D cell cultures is a well-known technique. However, in 2D cell cultures the cells lack many of the signals they usually receive in vivo. In fact, most of the mechanisms that rely on the organization of the tissue itself, and also mechanical and biochemical stimuli or interactions between cells are missing in 2D cell cultures. This inevitably leads to the fact that the observed cells have a phenotype different from that actually expressed in vivo. This lack of in vivo-like-physiology is the reason why complex systems such as animal models are used in the first place, although these models are still not satisfactory as described above.

This is why the development of new models that are more representative of human physiology (or more generally of in vivo physiology) is necessary in order to promote the research and development of molecules, new drugs and/or treatments. The new models should both minimize development costs and increase the chances of success of the molecules, new drugs and/or treatments to enter clinical trials.

The so called three-dimensional (3D) cell culture models are a promising advance in this regard. In the research and development of molecules, new drugs and/or treatments, 3D cell cultures represent more accurately human in vivo conditions. This allows a more effective evaluation of the performance of a molecule, drug and/or treatment from the preliminary phases of testing (i.e. first phases).

The so called “spheroids” (or spheroid cell cultures) are an example of promising 3D cell culture models. Spheroids are aggregates of cells that are formed by self-assembly when intercellular interactions are favored over their adhesion to a substrate. In other words, cells tend to aggregate together when they are placed in an environment estranged of their natural environmental conditions. The cells prefer to stick together than to attach to a substrate or support. Such spheroid 3D cell cultures allow a better reproduction of in vivo conditions where cells are naturally included in a three-dimensional structure composed of extra cellular matrix and other cells.

It has been shown that the cells of a spheroid preserve their initial phenotype. For example, stem cells keep their ability to differentiate. Further, spheroids enable cultivation of different types of cells in order to form organoids or mini-organs. A spheroid may also be used as a model of an avascular tumor and allows to test treatments by taking into account the architecture of such a tumor formation.

There are several techniques to induce the aggregation of cells to form spheroids. Most of them essentially rely on preventing the cells from adhering to a substrate. For example, one technique is based on surrounding the cells with a non-sticky (or non-adhesive) surface to cells. Another technique is based on keeping the cell culture medium constantly agitated [cf. Liu, D., Chen, S., & Win Naing, M. (2021)—A review of manufacturing capabilities of cell spheroid generation technologies and future development, Biotechnology and Bioengineering, 118(2), 542-554].

It is important to conduct experiments on a large number of spheroids that have essentially the same size in order to gain representative statistical results. There are two technologies that attempt to allow both size control and high throughput production of spheroids: the so called “hanging drop array technology” and the “microwells technology”.

The first technology consists in hanging drops of a few microliters under a plate. The cells contained in the drops sediment have no support to attach to and thus move more or less freely in the drops where they form a spheroid [cf. Kuo, C. T., Wang, J. Y., Lin, Y. F., Wo, A. M., Chen, B. P., & Lee, H. (2017)—Three-dimensional spheroid culture targeting versatile tissue bioassays using a PDMS-based hanging drop array, Scientific reports, 7(1), 1-10]. This technique shows good reproducibility performances, but it is challenging to change the culture medium. Furthermore, handling is difficult because of the very small volumes used in this technique.

The second technology uses microwells that comprise non-adhesive cavities of a few micrometers to millimeters in which cells can sediment and form a spheroid aggregate. This technique is generally easier to handle than hanging drops technology and shows similar performances in terms of spheroids production. However, disturbances and/or turbulences in the culture medium can cause spheroids to be ejected from their respective microwell. This may lead to the loss of valuable spheroids or to fusion of different spheroids together which both is problematic. Another problem of this technique is the loss of spheroids when the culture medium is changed. More generally, this technique is weakened by non-reproductive experiments and imprecise statistical analysis. All in all, the microwells technology is very sensitive to disturbances which makes this technique difficult on a large and reproductive scale.

Some state of the art documents tried to overcome above problems.

US2018264465A1 discloses a device for aggregating cells comprising at least one cavity, wherein said cavity comprises a plurality of microwells for receiving at least one cell, wherein each said well comprises a vertical sidewall and a curved bottom. To change the medium it is necessary to gently pipette against a side wall in order to minimize the loss rate of aggregated cells. This is incompatible with automated processes using robotized pipetting methods. High-throughput studies are drastically compromised. Furthermore, loss and fusion of spheroids remain problematic when an experiment requires multiple medium changes.

U.S. Pat. No. 9,790,465B2 discloses a spheroid cell culture article including a frame having a chamber including: an opaque side wall surface: a top aperture: a gas-permeable, transparent bottom: and optionally a chamber annex surface and second bottom, and at least a portion of the transparent bottom includes at least one concave arcuate surface. Methods of making and using the article are also disclosed. However, the materials used are rigid and require coating with another polymer in order to be used. As a consequence, the Young's modulus felt by the cells of the spheroid is generally too high which leads to alterations in gene expression. Also, fusion of spheroids and medium changes remains problematic.

US2021301237A1 discloses a well for cultivating biological material having a top opening and a bottom area including an internal edge. More precisely, the disclosure describes how to fabricate microwells within a main-well. Both spheroid-loss and spheroid-fusion remain problematic. Further, the disclosure describes how to extract medium by pipetting from an adjacent location to the microwells. Yet, consequences of pipetting from and adjacent location is that a significant portion of medium remains in the microwells, thus requiring at least multiple washing steps to remove cell waste. Further, it is not possible to remove dead cells that float in and over the microwells.

The state of the art shows that there is a demand for technologies that enable the production of spheroids in a reproducible and high-throughput manner. A key challenge is to provide a device and/or a method wherein the spheroids are preserved over long periods of time. Other problems yet to be solved are how to change media without spheroid loss, how to properly eliminate dead cells and/or how to transfer spheroids to another culture environment.

### SUMMARY

The present invention improves the situation.

Accordingly, the object of the present invention is cell culture device comprising at least one microwell comprising a top portion including a top opening for introducing cells, a bottom portion for cultivating said cells and a side wall joining said top portion to said bottom portion for guiding cells from said top opening to said bottom portion, wherein said top portion and said side wall are integral and made of a first material non-adhesive to cells and said bottom portion is made of a second material adhesive to cells.

According to an embodiment, said top portion is a first end face of said microwell, said microwell comprises a second end face opposite to said first end face, which second end face comprises a bottom opening, and said bottom portion is formed by a base made of said second material with said second end face resting on said base.

According to another embodiment, said bottom opening is arranged above said bottom portion in an overlapping manner.

According to another embodiment, said side wall is an inclined surface which connects the first end face to the second end face in order to guide cells deposited on said inclined surface substantially towards a coplanar level of the second end face by gravity so that the cells adhere to the bottom portion thus allowing their culture in order to form spheroids.

According to another embodiment, said bottom portion comprises a layer of functionalized extracellular matrix protein.

According to another embodiment, said first material is chosen in the group consisting of polyethylene glycol, a polyurethane polymer, polyamides, polyvinyl alcohol, polyethylene oxide, polypropylene oxides, polyethylene glycol, polypropylene glycol, polytetralethylene oxide, polyvinylpyrrolidone, polyacrylamide, polyhydroxyethyl acrylate, polyhydroxyethyl methacrylate, and mixtures thereof, PDMS and silicone-based materials. According to another embodiment, said second material is glass.

According to another embodiment, a plurality of microwells are arranged together in a seeding well.

According to another embodiment, said seeding well is arranged in a microwell.

According to another embodiment, the cell culture device further comprises a plurality of macrowells in which at least a seeding well is arranged.

According to another embodiment, the microwell is shaped in the form of a funnel.

Another object of the present invention is a method for cultivating spheroids, comprising the step of introducing cells and medium into a microwell of a device of the invention, letting the cells sediment to the bottom of said microwell by gravity and cultivating the cells at least for 3 hours, preferentially for 24 hours, more preferentially for 48 h, in order to enable said cells to aggregate into spheroids.

According to an embodiment, the method of the invention further comprises a step of adding cells of a different type from the cells of each spheroid in order to provide a co-culture of two different type of cells.

Thus, the object of the invention is a cell culture device comprising one microwell or an array of microwells, for cells, which rests on a base. The microwells are formed only by one piece (block of material) and have a first face which rests on the base and a second face substantially opposed to the first face, as well as a cavity having an opening at the level of the second face and an opening to the base at the level of the first face. The two openings are connected by an inclined plane (wall), which guides cells towards the bottom of the microwell, due to the surface properties of the inclined plane. Cells self-aggregate into a sphere like shape. The cells at the base of the sphere are in contact with the first opening, i.e., the one at the level of the first face and in contact with the base and get attached to the first opening point (area). The attachment of the cells to the opening area precludes the spheroid assembly to move when the medium is exchanged or the plate is moved, avoiding that they are mixed or fuse.

A second cell type can be added after the cell aggregate, spheroid, is formed. By gravity and due to the inclined planes, that form the walls of the microwells, the second cell type is guided towards the spheroids which were previously formed inside the microwells. This allows the second cell type to be in contact with the first cell type, forming a co-culture system, and interactions between them.

In one aspect, the present disclosure provides a cell culture device comprising at least one microwell comprising a top portion including a top opening for introducing cells, a bottom portion for cultivating the cells and a side wall joining the top portion to bottom portion for guiding cells from the top opening to the bottom portion, characterized in that the top portion and the side wall are integral and made of a first material non-adhesive to cells and the bottom portion is made of a second material adhesive to cells.

In some embodiments, the top portion is a first end face of the microwell, the microwell comprises a second end face opposite to the first end face, which second end face comprises a bottom opening, and the bottom portion is formed by a base made of the second material with the second end face resting on the base.

In some embodiments, the bottom opening is arranged above the bottom portion in an overlapping manner.

In some embodiments, the side wall is an inclined surface which connects the first end face to the second end face in order to guide cells deposited on the inclined surface substantially towards a coplanar level of the second end face by gravity so that the cells adhere to the bottom portion thus allowing their culture in order to form spheroids.

In some embodiments, the bottom portion comprises a layer of functionalized extracellular matrix protein.

In some embodiments, the first material is chosen in the group consisting of polyethylene glycol, a polyurethane polymer, polyamides, polyvinyl alcohol, polyethylene oxide, polypropylene oxides, polyethylene glycol, polypropylene glycol, polytetralethylene oxide, polyvinylpyrrolidone, polyacrylamide, polyhydroxyethyl acrylate, polyhydroxyethyl methacrylate, and mixtures thereof, PDMS and silicone-based materials.

In some embodiments, the second material is glass.

In some embodiments, a plurality of microwells are arranged together in a seeding well.

In some embodiments, the seeding well is arranged in a microwell.

In some embodiments, the cell culture device comprises a plurality of macrowells in which at least a seeding well is arranged.

In some embodiments, the microwell is shaped in the form of a funnel.

In another aspect, the present disclosure provides a method for cultivating spheroids, comprising the step of introducing cells and medium into a microwell of a device herein, letting the cells sediment to the bottom of the microwell by gravity and cultivating the cells at least for 3 hours, preferentially for 24 hours, more preferentially for 48 h, in order to enable the cells to aggregate into spheroids.

In some embodiments, the method further comprises a step of adding cells of a different type from the cells of each spheroid in order to provide a co-culture of two different type of cells.

### BRIEF DESCRIPTION OF THE DRAWINGS

Other features and advantages of the invention will stand out and/or become clear upon reading the present description, which comprises specific examples given in an illustrative and non-limiting manner, as well as from the figures in which;

FIG. 1 shows a side section view of a device according an embodiment of the invention;

FIG. 2 shows a top view of the device of FIG. 1;

FIG. 3 shows a cross-section view of a device according to another embodiment the invention including dimensions;

FIG. 4 shows a cross-section view of a device according an embodiment of the invention in the presence of a medium comprising cells;

FIG. 5 shows the cross-section view of the of FIG. 4 in the presence of a medium comprising cells after sedimentation of cells;

FIG. 6 shows a birds-eye view of a mold used to shape a device according to an embodiment of the invention;

FIGS. 7(a)-(c) shows a flowchart of a method to manufacture a device according to an embodiment of the invention;

FIG. 8 shows a three pictures of the formation spheroids in a device according to an embodiment of the invention, respectively after 3 hours, 24 hours and 72 hours;

FIG. 9 shows a schematic of the cross-section from a macrowell according to an embodiment of the invention;

FIG. 10 shows a top view of a 24-well plate according to an embodiment of the invention;

FIG. 11 shows a top view of a microstructured hydrogel according to an embodiment of the invention;

FIG. 12 shows a cross section with dimensions of two adjacent microwells according to an embodiment of the invention;

FIG. 13 shows pictures of a cell culture with a device according to the invention compared to a device of the state of the art;

FIGS. 14(a)-(b) shows a scheme of the addition of second type of cells to a first cell type constituting a spheroid: and

FIGS. 15(a)-(g) shows pictures of images taken of spheroids before and after adding a second type of cells.

### DETAILED DESCRIPTION OF THE EXAMPLE EMBODIMENTS

The present invention provides a technique that ensures that the spheroids remain in their respective microwell during experimental manipulations, including medium exchange and/or the addition of test molecules. Thus, the invention allows calibrated, high-throughput, automatable production of spheroids. Experiments on the spheroids regarding research and development of molecules, new drugs and/or treatments can be performed directly on the cell culture device of the invention, i.e. without the need to transfer the spheroids to another environment.

The present invention comprises a three-dimensional structure made of a biocompatible and optically transparent material (hydrogel or biocompatible elastomer) allowing the autonomous and reproducible aggregation of cells to form spheroids. The three-dimensional structure comprises at least one microwell, generally several microwells, molded on a flat substrate. The flat substrate may be a glass plate for example.

A feature of the invention is that the three-dimensional structure is made of a material that is non-adhesive to cells, and that the flat substrate is made of a material that is adhesive to cells. According to a preferred embodiment, the general aspect or design of a device from the invention is in the form of a plate, comparable to known multi-well plates.

Each microwell from a three-dimensional structure has inclined side walls and has an opening at the bottom so that the ground from each microwell is a part of the flat substrate on which the three-dimensional structure is molded. This bottom opening from each microwell provides access to an adherent surface for the cells to which cells can attach. Once cells agglomerate the cells attach to the adherent surface forming an anchor point (or anchor surface) for the spheroid. When cells initially attach to the anchor point, the aggregation of the cells at the bottom of said microwells is enabled. During the self-assembly of the spheroid and/or once the spheroid is formed, the spheroid remains in place even if the culture medium is disturbed, e.g. by removing/replacing medium or if the entire device is shaken.

The anchor point may be functionalized with extracellular matrix proteins to promote cell adhesion and activate certain signaling pathways within the cells. A non-limiting list of examples of such proteins includes: Fibronectin, Collagen, Laminin, Fibrinogen, Matrigel.

More generally, all the materials used for the device of the present invention are substantially optically transparent. Further, the spheroids cultivated with the device of the present invention are placed and anchored in the same focal plane, which allows easy observation by optical imaging. In addition, each spheroid is maintained in the center of the bottom of its respective microwell, or more generally in the center of its respective microwell. This allows individual and reproducible monitoring of the spheroids evolution by automated image acquisition.

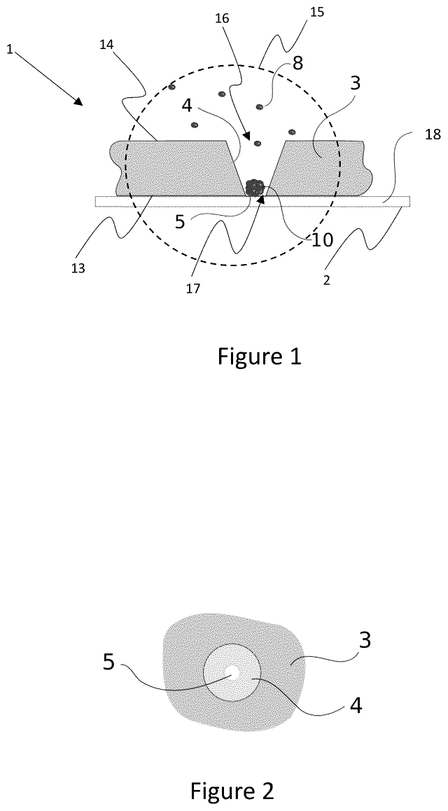

FIGS. 1 and 2 show a cross section view and a top view respectively of a device according to an embodiment of the invention.

A cell culture device 1 for cultivating cells according to an embodiment of the invention comprises a base 2, which preferentially presents itself as a flat plate. More generally, the base 2 may be a flat substrate made of a material 3 that does not allow cell adhesion. The material 3 is molded onto the base 2. The material 3 comprises one or more microwells 15. According to the invention, the bottom of each microwell 15 comprises an opening 17 that provides access to the base 2. Each microwell 15 is preferably of conical shape, more preferentially shaped as a funnel, and the opening 17 at its bottom is arranged in the center of the corresponding microwell. Each microwell 15 comprises a side wall 4. The side wall 4 is inclined to allow cells 8 to settle at the bottom of the microwell 15 where they can aggregate and form a spheroid 10. The opening 17 at the bottom of the microwell 15 provides access to a bottom portion 5 of the microwell usually constituted of a part of the surface of base 2 to which the cells 8 can adhere. The surface where the cells can adhere thus forms an anchor point for the cells, and ultimately to the spheroid 10. A plurality of microwells 15 can be assembled together, for example on a base plate 2 or in the well of a multi-well plate, to allow the formation of several spheroids 10 under the same experimental conditions (cf. below).

More generally, FIGS. 1 and 2 show a cell culture device 1 comprising at least one microwell 15 having a top portion 14. The top portion 14 includes a top opening 16 for introducing cells 8. The microwell 15 also comprises a bottom portion 5 for cultivating the cells and a side wall 4 joining said top portion 14 to the bottom portion 5 for guiding cells from the top opening 16 to the bottom portion 5. According to the invention the top portion 14 and said side wall 4 are integral (i.e. made of one piece) and made of a first material 3 non-adhesive to cells and the bottom portion 5 is made of a second material 18 adhesive to cells.

FIGS. 1 and 2 show the top portion is a first end face 14 of the microwell 15 which may be at least partially planar. The microwell 15 further comprises a second end face 13 opposite to said first end face which may also be at least partially planar. The second end face 13 is arranged on the base 2. Typically, the second end face 13 rests upon the base 2. The second end face 13 comprises a bottom opening 17 so that the bottom portion 5 is formed by the base 2 that is made of the second material 18 to which cells can adhere.

According to a preferred embodiment of the invention, the bottom opening 17 is arranged directly above the bottom portion 5. In other words, the bottom opening 17 and the bottom portion 5 of the microwell 15 are overlapping.

The first material 3 used for the device of the invention is preferably a hydrophilic and transparent polymer that does not allow cell adhesion, such as polyethylene glycol (PEG), preferably a hydrophilic polymer capable of forming a hydrogel, preferably a polymer that can be cross-linked by photopolymerization, preferably a hydrogel with a Young's modulus suitable for the cells used, preferably less than 100 kPa. Further the first material according to the invention is moldable into a three-dimensional structure as detailed later. Following polymers may be used as first material non-adherent to cells: poly(ethylene glycol) or polyethylene glycol, a polymer of the polyurethane family, polyamides, polyvinyl alcohol, poly(ethylene oxide), polypropylene oxides, polyethylene glycol, polypropylene glycol, polytetralethylene oxide, polyvinylpyrrolidone, polyacrylamide, poly(hydroxyethyl acrylate), poly(hydroxyethyl methacrylate), and mixtures thereof. Hydrophilic elastomers such as Polydimethylsiloxane (PDMS) or silicone-based materials may also be considered.

The second material 18 is made of a transparent and rigid material that can be glued to the bottom of a multiwell plate, preferably the second material is glass. The second material 18 preferentially comprises an upper side that has undergone a surface treatment in order to promote adhesion of the first material 3 onto the second material 18. Preferentially, the second material has undergone a surface treatment so that the first material 3 adheres to it covalently, for example by silanization.

The bottom portion 5 at the bottom of the microwell 15 is preferentially coated with proteins that promote cell adhesion. Such proteins may be contained in the culture medium 7. Examples may come from animal serum. Such proteins also may be components of the extracellular matrix such as collagen or fibronectin.

The device of the invention comprises well plates containing hydrogel-based microwell structures specially designed for the spontaneous formation of uniform spheroids. The anti-adhesive properties of the hydrogel facilitates rapid self-assembly of cell aggregates into compact spheroids.

The adhesive bottom surface 5 of each microwell 15 anchors the spheroids 10 in place. This prevents the loss of spheroids during typical manipulations (medium exchange, drug addition, etc.), and makes the location of each spheroid predictable, as the spheroid remains attached throughout the cell culture timeline. As the hydrogel is optically transparent, it has a refractive index close to the one of water (1.33) it is fully compatible with conventional microscopy.

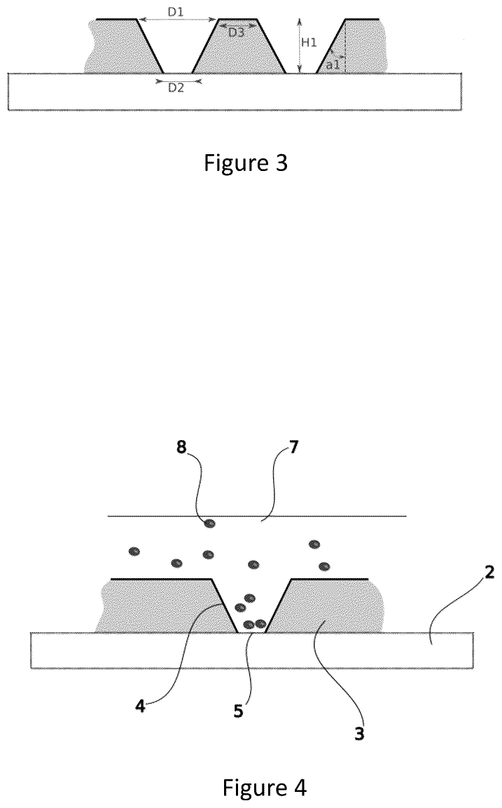

FIG. 3 shows cross-section with dimensions of adjacent microwells of a three-dimensional structure according to the invention.

The microwells 15 have a top opening 16 marked D1 between approximately 50 μm and 1 cm, preferably between 100 μm and 2 mm. The top opening 16 may be of diverse shape such as circular, hexagonal or octagonal for example. The bottom opening 17 marked D2 at the bottom of the microwell is between approximately 1 μm and 1 mm, preferably between 50 μm and 200 μm. The bottom opening may be of diverse shape such as circular, hexagonal or octagonal for example. Preferentially, the bottom opening is circular and has a diameter between approximately 1 μm and 1 mm, preferably between 50 μm and 200 μm. The spacing between two adjacent microwells, marked D3, is between approximately 0.1 μm and 500 μm, preferably less than 50 μm. According to an embodiment, there is no spacing between adjacent microwells. When present, the spacing D3 should be minimized to avoid cells remaining between the microwells. This usually ensures that the maximum number of cells are seeded at the bottom of each microwell to form a spheroid, rather than between the microwells. The height of the microwells marked HI is between approximately 50 μm and 5 mm, preferably between 500 μm and 2 mm. The angle with respect to the vertical of the walls marked al of the microwells is between 0°) and 90° (degrees), preferably between 10° and 50°. The walls might be continuous from top to bottom, or might have two inclination angles, where close to the bottom of the microwells the inclination of the wall is less steep than the top part, to better accommodate the spheroid and induce a perfectly spherical shape, rather than a conic one.

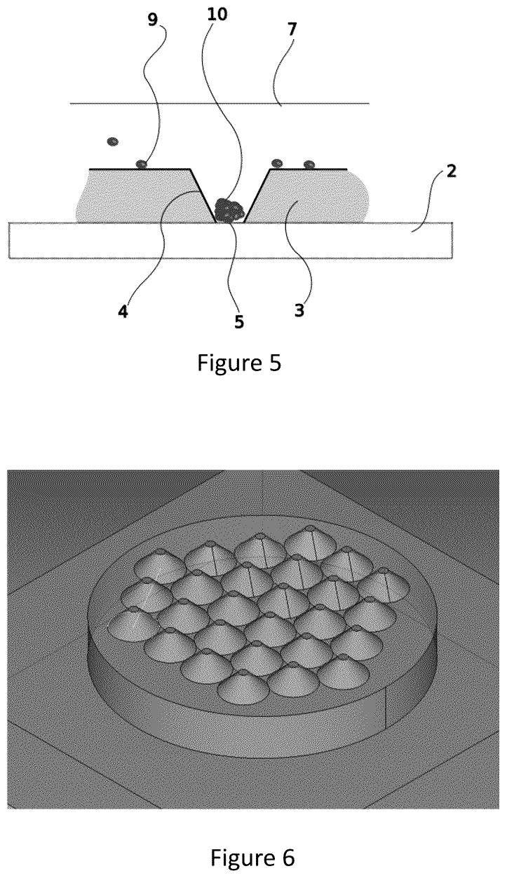

FIGS. 4 and 5 respectively show a cross-sectional view of a microwell molded in a larger well into which a suspension of cells has been introduced, and a cross-sectional view of the same microwell after said cells have sedimented and formed a spheroid-shaped aggregate.

A cell culture medium 7 containing suspended cells 8 is introduced above one or more microwells 15, for example inside a well of a multi-well plate or in a petri dish. Under the influence of gravity, the cells 8 sediment and settle either between the microwells 9 or at the bottom 5 of a microwell 15. The first material 3 according to the invention does not allow cell adhesion, i.e. it is non-adhesive to cells. The first material 3 thus deprives the cells of finding a proper support. As a consequence, the cells 8 establish links between each other and form three-dimensional aggregates, which are qualified as spheroids 10. On the other hand, the cells of the spheroid 10 in proximity of the opening 17 at the bottom of the microwell 15 establish connections with the second material 18 of the base 2. Consequently, the spheroids 10 are anchored to the bottom 5 of the microwell 15.

Cells can be left to sediment just by gravity, or the cell culture plates can also be centrifuged to speed up the sedimentation of the cells, which leads to a faster formation of the spheroid.

The seeded cells must be able to form bonds with each other and with proteins that can adsorb to the base 2 made of second material 18. Once the spheroid(s) is formed, the culture medium 7 can be changed to remove cells that have not entered the microwells, and thus without an attachment point, without risking disruption of the spheroids that are anchored to the substrate. According to a preferred embodiment, the cell culture device 1 is transparent. Accordingly, it is possible to image the spheroids by microscopy. Further, the immobilization of the spheroids in each microwell 15 by means of the anchor points according to the invention makes it possible to monitor individual spheroids over time, as they are kept in place and there is no movement and consequent merging of the cell aggregates.

FIG. 6 shows a birds-eye view (or perspective view) of a possible design of a mold used to shape a hydrogel of the invention and then insert it into a 96-well plate. This is a positive mold.

In this embodiment, the manufacture of the cell culture device 1 of the invention first comprises the fabrication of a rigid elastomer mold. For this, a first mold of rigid material, for example resin or metal, is produced using a method such as 3D printing or CNC milling. The first mold can be a positive (i.e. with pillars protruding in the shape of the microwells 15) or a negative (i.e. with cavities in the shape of the microwells 15). An elastomeric part, for example PDMS, is molded onto the first rigid mold. If the first rigid mold is negative, the second elastomeric mold is positive and can be used to mold the first material 3. Otherwise, the elastomeric part is used to mold another (third) elastomeric mold that will be a positive and which can be used to mold the first material 3.

Preferentially a negative Stainless Steel mold machined using high resolution CNC milling is used to originate a second elastomeric mold.

The elastic property of the mold used to fabricate the final cell culture device ensures that when molding the microwells, preferentially using a hydrogel such as PEG, the mold deforms and compensates for the differences in height from pillar to pillar ensuring that there is always an opening at the bottom of the microwell. The same is not possible with a rigid mold, as the variability of the height of the pillars with the shape of the microwells can differ slightly from microstructure to microstructure, which ultimately leads to microwells that having respective contact points, and to some other that do not have such contact points.

If PDMS is used as the mold, a 10:1 by weight mixture of monomers and crosslinker is prepared and mixed. Bubbles are removed in a vacuum chamber. The mixture is poured onto the previous mold and then cured at 80° C. for at least 2H. Before molding PDMS onto another PDMS mold, the mold is exposed to a radical ozone plasma to ensure that the new part does not polymerize with the mold. Preferentially, only one PDMS based mold is produced, as the master mold has a negative shape (carved microwells).

Once the mold is ready, the three-dimensional structure made of the first material 3 can be prepared and shaped as described.

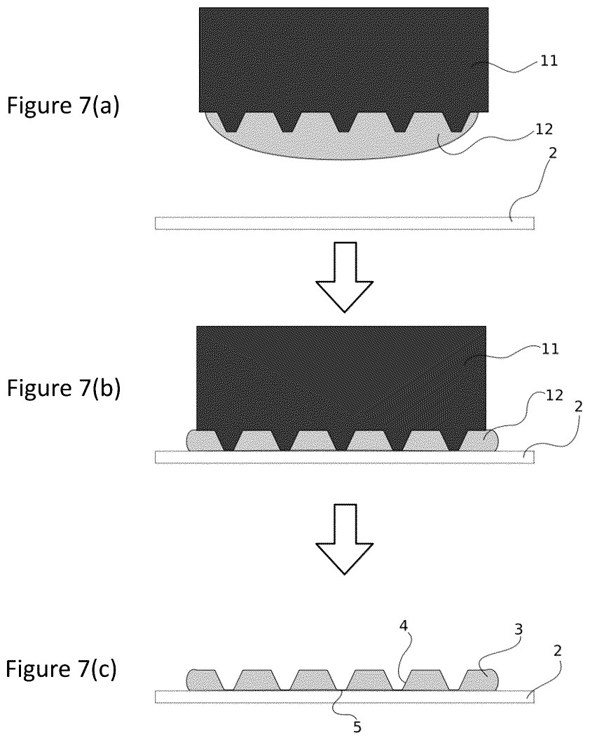

FIG. 7 shows a flowchart of a method to manufacture a device according to an embodiment of the invention. FIG. 7(a) shows a side view of a mold (11) having a shape designed to provide a plurality of microwells when a drop of hydrogel (12) solution is deposited on the mold: FIG. 7(b) shows a side view of the same mold (11) when pressed against a flat substrate (2), also called base, in order to provide the desired shape to the hydrogel solution; and FIG. 7(c) shows a side view of the hydrogel deposited and attached to the flat substrate (2) after being cured and after the mold (11) being removed.

The first material 3 used in the present embodiment is a four-armed PEG-SH and four-armed PEG-norbomene copolymer, both with a molecular weight of 10 kDa. It is shaped by molding using an elastomer mold 11 made of PDMS. Both polymers are dissolved in distilled water in equal amounts to reach a final polymer mass concentration of 6.4%. A photoinitiator that generates radicals when exposed to UV, such as lithium phenyl-2,4,6-trimethylbenzoylphosphinate (LAP), is introduced for a final concentration of 0.2%. This provides a solution 12. An approximately 10 μL to 100 μL drop of solution 12 is placed on the patterned side of the PDMS mold 11, cf. FIG. 7a. Once the entire surface of the mold 11 is covered, it is deposited on a flat substrate, i.e. base 2 previously functionalized with a molecule that allows the material to adhere, here a silanized glass plate. The elastomer mold 11 can be pressed against the flat substrate to ensure that both the mold 11 and the base 2 are in direct contact. The first material 3 is then exposed to ultraviolet light (365 nm) suitable for activating the photoinitiator. As a consequence, the PEG-SH and PEG-norbomene copolymer crosslink with each other. This forms a three-dimensional gel structure. The mold 11 is then removed and the cross-linked material 3 remains attached to the base 2 leaving openings 17 at the bottom of each formed microwells 15. The base 2 can be glued to the bottom of a multi-well plate adapted to the dimensions of the three-dimensional structure of the invention (i.e. gel) or inside a petri dish so that it can be seeded with cells. Usually, the gel is kept in distilled water to avoid dehydration before its final use.

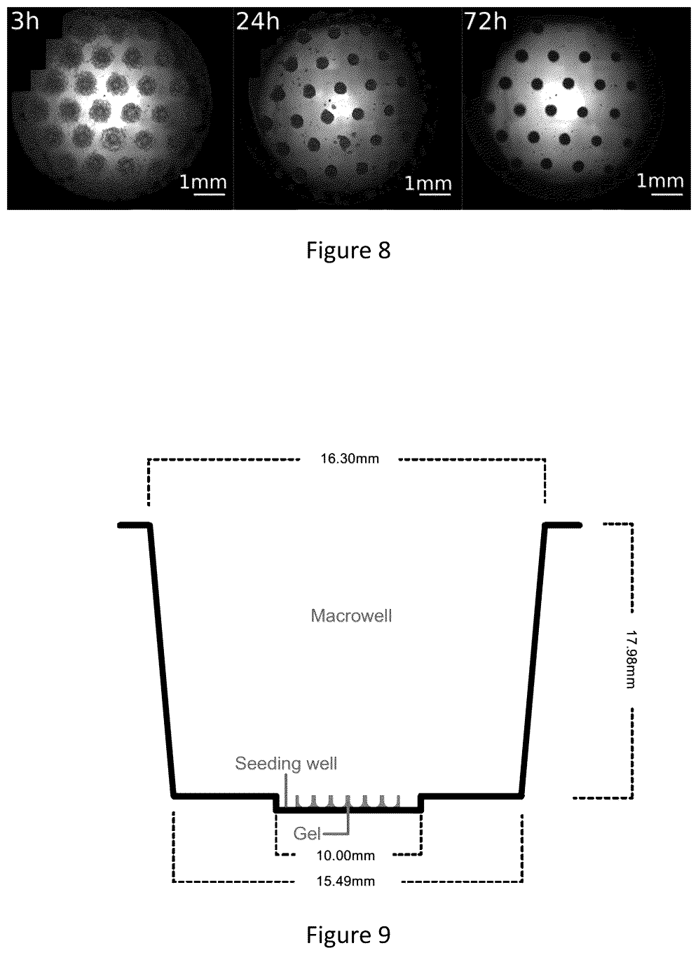

FIG. 8 shows formation of mouse mesenchymal stem cells (MSCs) spheroids in PEG microwells. Sedimentation is visible after 3 h of incubation, and the cells aggregate into spheroids within 24 h. By changing the culture medium every 24 hours, spheroids can be maintained for several weeks in culture The porous properties of the hydrogels make it easier for the nutrients to reach the spheroids.

The three pictures were taken under following condition: A monoculture of MSCs (mouse mesenchymal stem cells) is cultivated in a cell culture flask in RPMI (Roswell Park Memorial Institute Medium) supplemented with 10% FBS (Fetal Bovine Serum) and 1% penicillin/streptomycin. When the cells are at confluence, they are detached with a trypsin solution, recovered in medium and counted with a hemocytometer. The final cell concentration is calculated according to the number of microwells in the well and the number of cells per spheroid. For one well of a 96-well multiwell plate containing 27 microwells, 300 μL of medium containing 900,000 cells per mL should be introduced to produce spheroids of 10,000 cells each. Before seeding the cells on the gel, the gel is sterilized with UV light for 30 min, then the distilled water used for its preservation is exchanged with culture medium identical to that used for the cells. The gel is then incubated at 37° C. for 30 minutes. The medium is removed from the well and the cells are subsequently introduced into the well. The culture plate is then incubated at 37° C. for the duration of the culture. The medium is then renewed at least every two days, preferably every day.

FIG. 9 shows a schematic of the cross-section from a macrowell according to an embodiment of the invention (not to scale).

According to an embodiment of the invention, a hydrogel (or gel) made of the first material 3 according to the invention can be deposited to the surface of a glass in the middle of a seeding well. A seeding well is arranged as a bowl containing a plurality of microwells. A seeding well has side walls at least as high as the microwells, preferentially higher so that medium and cells may be deposited directly into the seeding well, thus filling-up the microwells with medium and cells.

The seeding well comprises microwells 15 of the invention arranged therein. The seeding well itself is arranged in a larger macrowell as shown in FIG. 9. A macro well is arranged as a bowl containing at least one seeding well. A macro well has side walls higher than each seeding well. Generally, the macrowell has a top opening of approximately 15 mm to 40 mm, a bottom width of approximately 15 mm to 40 mm and a height of approximately 10 mm to 20 mm. The seeding well has a width of approximately 5 mm to 10.00 mm and a height of approximately 1 mm to 3 mm.

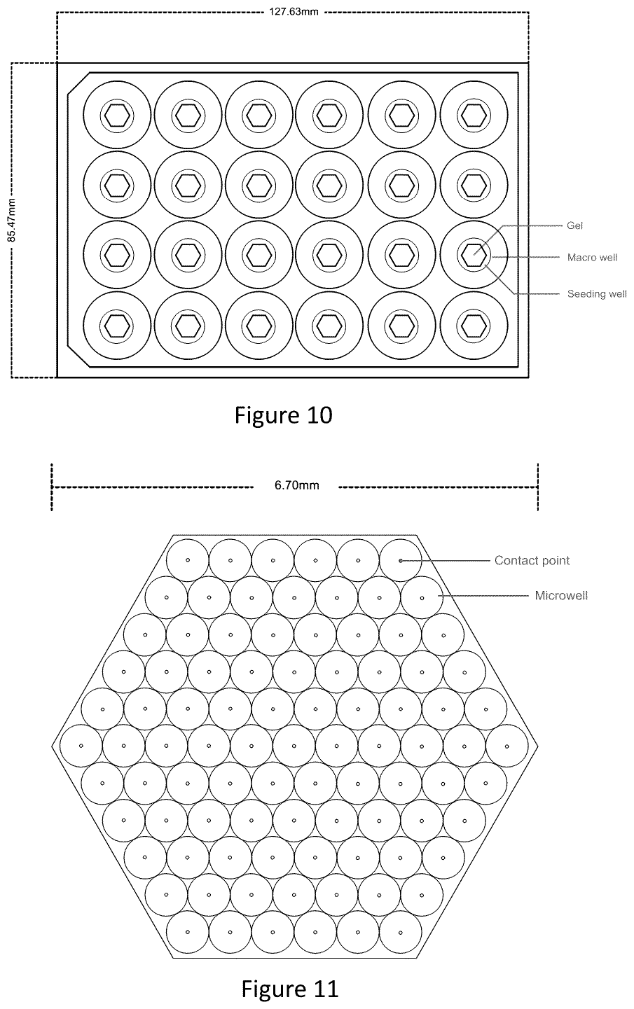

FIG. 10 shows top view of a 24-well plate of an embodiment of the invention.

According to an embodiment of the invention, a plate of approximately 120 mm to 130 mm×80 mm to 90 mm may comprise a plurality of macrowells, i.e. here 24. Accordingly, the plate also comprises a plurality of seeding wells, i.e. 24, each comprising a greater plurality of microwells 15 made of hydrogel (noted gel).

The seeding wells allow for a smaller volume of cell suspension to be used, as there is no need to cover all the area formed by the bottom of the macrowells, maximizing the number of cells that are used to form the spheroids and minimizing the number of cells that are lost.

The macrowell can simply be the well of the multi well plate, for instance for cases where the wells are smaller (ex. 10 mm wide for a 48 well plate, or 6 mm wide for a 96 well plate). In this case the number of cells is also optimized as the microstructured hydrogel with microwells ends up covering the majority of the area of the wells.

FIG. 11 shows a top view of a microstructured hydrogel according to an embodiment of the invention. In this embodiment, there are 91 microwells each having an anchor point (noted contact point). Accordingly, a plate of FIG. 10 comprising 24 macrowells may comprise 2184 microwells 15 according to the invention.

In other embodiments, the seeding well is not included. Accordingly, plates are used that have only a regular well. The gels are fabricated at the bottom of the regular well, without the presence of an indentation/smaller cavity (seeding well as described above).

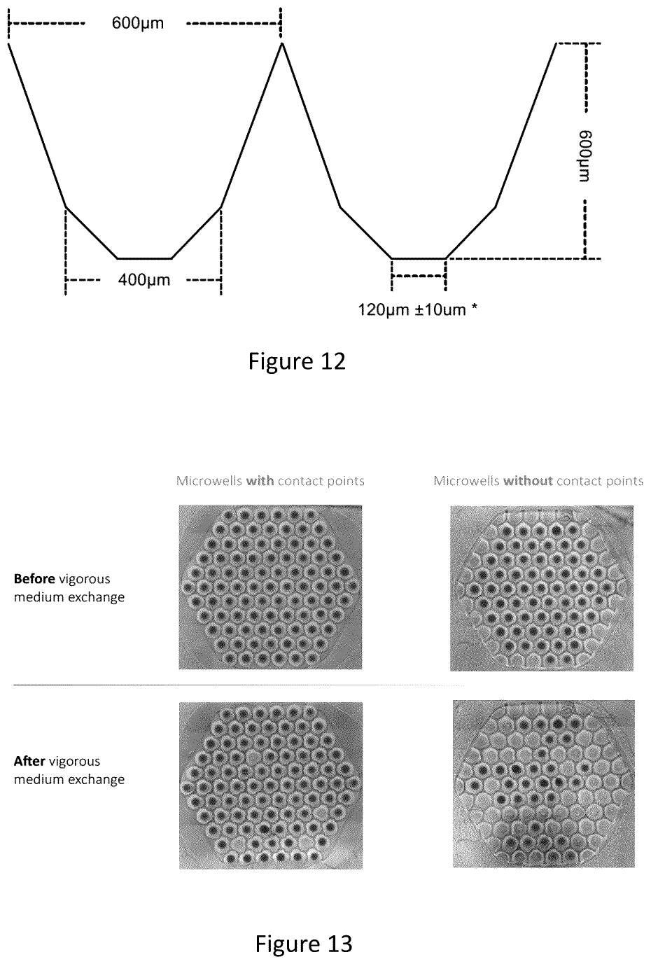

FIG. 12 shows a cross section with dimensions of two adjacent microwells according to an embodiment of the invention. Each design of a microwell according to the invention is customizable. The top opening in this embodiment has a width and μm of 600 μm and a bottom portion of a width of approximately 110 μm to 130 μm. The bottom portion comprises the anchor point. Inclined walls may differ in angle degrees as shown in FIG. 12 over a width of 400 μm.

FIG. 13 shows comparison of detached spheroids between a device having anchor points according to the invention vs. a gel without anchor points after vigorous medium exchange.

The size of the spheroids can be tuned by changing the number of seeded cells. The upper right side of FIG. 13 shows a picture of a device (not according to the invention) having a plurality of state of the art microwells. The upper left side of FIG. 13 shows a picture of a device according to the invention having a plurality microwells 15 with a bottom portion providing an anchor point (or contact point). Both the upper right and left side pictures show spheroids. Both pictures were taken before vigorous medium exchange. The bottom part of FIG. 13 shows pictures of both devices after vigorous medium exchange: Most of the spheroids were lost in the device of the state of the art, while almost all the spheroids were intact and in place in the device according to the invention.

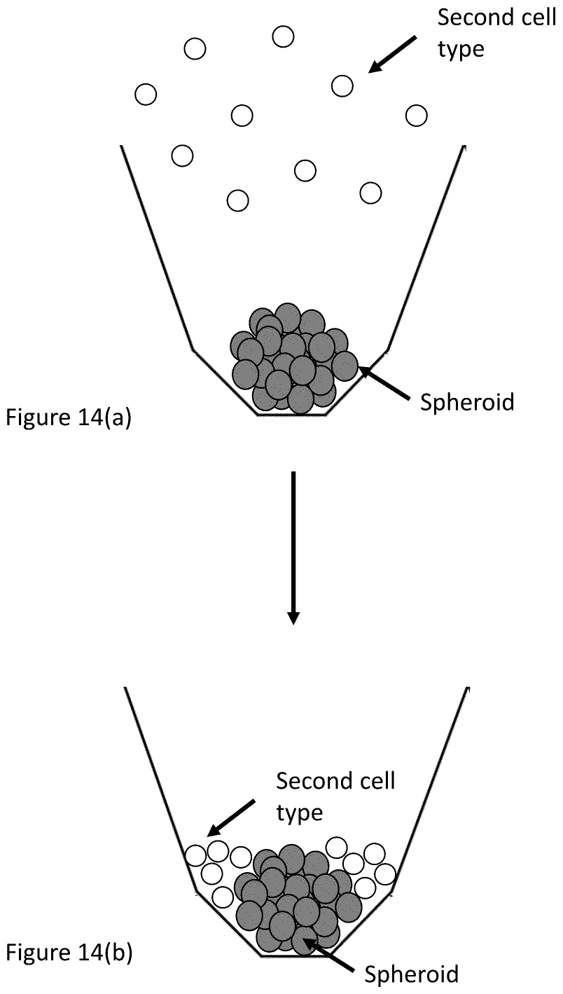

FIG. 14 shows a scheme of a protocol for the addition of second type of cells to a first type of cells.

According to an embodiment, the device of the invention is used for co-culture of at least two different types of cells. The first type of cells being the ones constituting the spheroid, and the second type added to the microwells after the aggregation and compaction phases, cf. FIG. 14(a).

The spheroids may be formed by one type of cells or by a mix of different cell types to provide more relevancy to a biological model.

The second type of cells should be added about 24 to 48 hours after the first type of cells was seeded, thus allowing for aggregation and compaction of the spheroid, and further avoiding the first and second cell types to be mixed in the spheroid.

The second type of cells are guided, towards the microwells area, where the first type of cells is located. This maximizes the number of spheroids put in contact with a second type of cells, cf. FIG. 14(b).

The second type of cells are generally left to sediment for 1 to 2 hours before moving the plate.

The contact point/attachment area of the microwells prevents that the first cell type, i.e. the spheroid, is removed from the microwells, while aspirating the medium before adding the second cell type.

The suspension of the second cell type should be well homogenized to ensure that when it is pipetted on the top of the first cell type the number of cells per microwell is approximately the same everywhere.

Alternatively, the plate can be centrifuged to speed up the sedimentation of the second cell type inside the microwells where the spheroid is located.

Centrifugation should be between 2 g and 200 g for 30 seconds to 2 minutes, preferably 100 g for one minute.

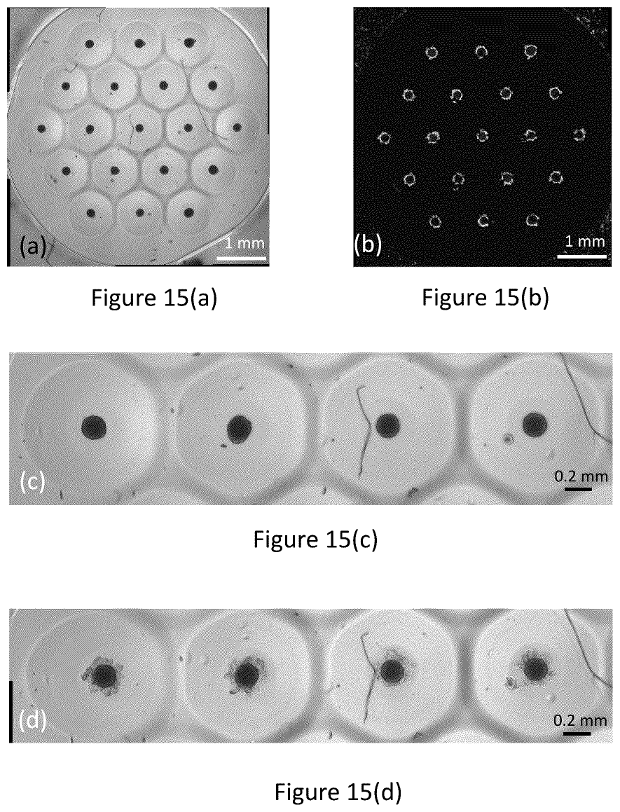

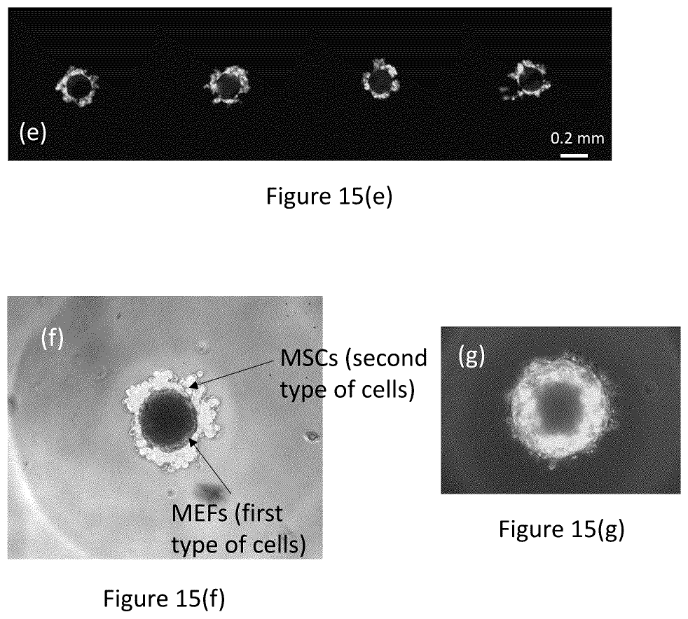

FIG. 15 shows pictures of images taken of the spheroids before and after adding a second cell type. In the present example, the spheroid is formed by Mouse Embryonic Fibroblasts and the second cell type are Mesenchymal Stem Cells, expressing a Green Fluorescent Protein (i.e.: Spheroid—MEF cells, second cell type—MSC cell expressing GFP).

More precisely, FIG. 15 shows bright field images and fluorescent images of the spheroids and second cell type. FIG. 15(a) shows Spheroids after 24 hours of being formed and FIG. 15(b) shows spheroids surrounded with the second cell type after 1 hour of sedimentation. FIG. 15(c) shows a zoom-in of four spheroids before the second cell type is added, FIG. 15(d) shows a zoom-in of four spheroids after the second cell type is added, and FIG. 15(e) shows a zoom-in of four spheroids after the second cell type is added where only the second cell type surrounding the spheroid is visible since the second cell type express GFP. FIG. 15(f) shows a composition image of bright field image of a spheroid with fluorescent image of the second cell type. FIG. 15(g) shows a composition image of bright field image of a spheroid with fluorescent image of the second cell type after 48 hours showing that the second cell type starts to cover the spheroid, as the second cell type proliferates.

Two different arrangements of microwells were used, namely a cell culture device with 19 microwells and a cell culture device with 91 microwells. The footprint used by both is approximately the same, which means that in the cell culture device that has 19 wells the spheroids are more spaced from each other.

The cell culture devices were fabricated according to the processes described in this invention: 20 μL of gel solution was added to a positive PDMS mold (fabricated form a negative master mold made of Stainless Steel and fabricated using high resolution CNC milling) with 19 microwells (in another embodiment 13 μL of gel solution was added to a 91 microwells mold, not shown). The molds were put in contact with a glass surface and cured for 1 minute using an LED light source (365 nm-70 mW/cm2). When all gels were molded and cured, they were submerged in medium and the plate was put into the incubator while the cell suspension was prepared, to ensure that the hydrogels are in equilibrium with the cell culture medium.

A confluent t-25 flask of MEFs was taken from the incubator, the medium was removed, washed with 0.5 ml PBS and 0.5 ml of TrypLE Express enzyme was added before the flask was placed back in the incubator for 3 minutes. After 3 minutes, the flask was taken out of the incubator and the TrypLE was diluted with 4.5 ml of DMEM culture medium. A cell-suspension of MEFs was prepared at a concentration of 950.000 cells per mL for the 19 microwell mold and 2.730.000 cells per mL for the 91 microwell mold. The plate was removed from the incubator and the medium was removed.

100 μL of cell suspension was added on top of the gels and the plate was transferred back to the incubator for 1 hour to let the cells sediment into the microwells. After 1 hour, an additional 2 mL of medium was added and the cells were left to aggregate in the incubator overnight. After 24 hours, three different concentrations of cell suspensions with MSCs were prepared (300) cells per microwell, 600 cells per microwell and 1200 cells per microwell).

A confluent t-25 flask of MSCs was taken from the incubator, the medium was removed, washed with 0.5 ml PBS and 0.5 ml of TrypLE Express enzyme was added before the flask was placed back in the incubator for 3 minutes. After 3 minutes, the flask was taken out of the incubator and the TrypLE was diluted with 4.5 ml of RPMI culture medium

100 μL of the MSC cell suspensions were added to the gels containing the 24 hours old MEF spheroids.

The plate was left in the incubator for 1 hour to let the cells sediment. Pictures were taken 1-2 hours after seeding the MSCs and 48 hours after showing the proliferation of the second cell type. The two cell types can be differentiated from each other because the MSCs express a Green Fluorescent Protein that the MEFs do not. The spheroids are identified by a black core, while the second cell type are the bright spots around the spheroid, as shown in FIG. 15.

The following is a non-limiting summary of the main aspects of the invention;
Cells are cultured in microwells inducing their aggregation and the formation of spheroids 10;
The microwells 15 comprise a first material 3 made of a biocompatible hydrophilic polymer that does not allow cell adhesion;
The bottom of the microwells 15 comprises a second material 18 which allows cell adhesion: in an embodiment the device of the invention comprises an opening arranged on a rigid base which allows the cell adhesion;
The microwells have sloping side walls to promote cell sedimentation within them;
The spheroids 10 formed within the microwells 15 are attached to an anchor point that holds them sufficiently so that they are not displaced when the culture medium is changed;
The material forming the first material 3 of the microwells 15 is shaped by molding and light curing;
The rigid substrate base 2 made of second material 18 is treated so that the first material 3 adheres covalently to it;
The molding process can be adapted to form said microwells in a multi-well plate
Both the first material 3 and the second material are optically transparent and allow in situ visualization of the spheroids 10;
Adhesion of spheroids 10 to an anchor point allows individual monitoring over a long period of time by regularly changing the medium without losing or displacing the spheroids;
The bottom 5 of each microwell can be functionalized with proteins, for example with animal serum or extracellular matrix to improve the adhesion of cells.
The size of the spheroids is reproducible and can be tuned just by adjusting the number of seeded cells.
According to an embodiment, the device of the present invention enables a method of co-culture of at least two types of cells, without any cell loss: 100% of the cells are conserved. In other embodiments almost none of the cells are lost, preferentially at least 95%, more preferentially over 98% of the cells are conserved (i.e. thus substantially without any cell loss).

The Inventors of the present invention have developed an innovative anchoring technology. The technique of the present invention enables to work with up to 5280 traceable spheroids per plate.

In other words the device of the present invention comprises a two layer system. A first bottom layer consisting of a support plate adhesive to cells, preferentially made of glass, and a top layer consisting of a hydrogel non adhesive to cells, and preferentially permeable to nutrients. The hydrogel and the support plate are in contact with each other. The hydrogel comprises funnel shaped microwells having an opening at their bottom end that gives access to the support plate. The bottom of each microwell thus is constituted by a portion of the support plate.

Consequently, at the bottom of each well: the glass support plate provides an anchoring point for cells. The anchoring point (sometimes referred to as contact point or support point) helps keeping the spheroids in each microwell during medium exchanges. Although the spheroids can move, they do not detach from the support plate with medium exchange flow. The movement of the anchored spheroids may be somewhat comparable to algae movement on the ground of the sea.

A method to cultivate spheroids in the device of the invention comprises;
Inoculating wells from the device of the invention with cells;
Cells wander/fall to the bottom of each well and make contact with the anchoring point;
Growing cells (e.g. 24-48 h): cell spheroids are formed;
Each cell spheroid adheres to the respective anchoring point of the corresponding well: they can move but do not detach with the medium exchange flow.

The device of the invention provides:
High reproducibility;
Predictable position: cells may be plated directly on the microstructured substrates;
Uniform size and shape of the cell culture, i.e. size controlled spheroids: the size of the spheroids depends only on the cell seeding concentration;
No loss of spheroids, especially no loss during medium exchange;
Compatibility with imaging technologies;
Traceability: cells are easy to locate and trace, i.e. cell aggregates are located in predictable positions enabling automated image acquisition.

According to other embodiments of the invention the device may comprise 6-, 12- or 24-well (macrowell) plates with glass bottom. Each macrowell may comprise 91 individual microwells where spheroids are assembled. The microwells are made of a three dimensional microstructured substrate such as hydrogel, preferentially biocompatible polyethylene glycol gel. Optionally, at the bottom the microwells can be coated with an ECM matrix to increase the adherence of the spheroid to the contact (attachment) point. When cells are added to the device of the invention, the initial stages of spheroid formation begins: including cell aggregation and spheroid compaction, followed by spheroid growth in the days following compaction due to cell proliferation. Further, according to an embodiment, once the spheroid is formed another type of cells may be added for co-culturing different types of cells.

What is claimed is:

1. A cell culture device comprising at least one microwell comprising a top portion including a top opening for introducing cells, a bottom portion for cultivating said cells and a side wall joining said top portion to said bottom portion for guiding cells from said top opening to said bottom portion,
characterized in that said top portion and said side wall are integral and made of a first material non-adhesive to cells and said bottom portion is made of a second material adhesive to cells.

2. The cell culture device according to claim 1, wherein said top portion is a first end face of said microwell, said microwell comprises a second end face opposite to said first end face, which second end face comprises a bottom opening, and said bottom portion is formed by a base made of said second material with said second end face resting on said base.

3. The cell culture device according claim 1, wherein said bottom opening is arranged above said bottom portion in an overlapping manner.

4. The cell culture device according to claim 1, wherein said side wall is an inclined surface which connects the first end face to the second end face in order to guide cells deposited on said inclined surface substantially towards a coplanar level of the second end face by gravity so that the cells adhere to the bottom portion thus allowing their culture in order to form spheroids.

5. The cell culture device according to claim 1, wherein said bottom portion comprises a layer of functionalized extracellular matrix protein.

6. The cell culture device according to claim 1, wherein said first material is chosen in the group consisting of polyethylene glycol, a polyurethane polymer, polyamides, polyvinyl alcohol, polyethylene oxide, polypropylene oxides, polyethylene glycol, polypropylene glycol, polytetralethylene oxide, polyvinylpyrrolidone, polyacrylamide, polyhydroxyethyl acrylate, polyhydroxyethyl methacrylate, and mixtures thereof, PDMS and silicone-based materials.

7. The cell culture device according to claim 1, wherein said second material is glass.

8. The cell culture device according to claim 1, wherein a plurality of microwells are arranged together in a seeding well.

9. The cell culture device according to claim 8, wherein said seeding well is arranged in a microwell.

10. The cell culture device according to claim 9, comprising a plurality of macrowells in which at least a seeding well is arranged.

11. The cell culture device according to claim 1, wherein the microwell is shaped in the form of a funnel.

12. A method for cultivating spheroids, comprising the step of introducing cells and medium into a microwell of a device according to claim 1, letting the cells sediment to the bottom of said microwell by gravity and cultivating the cells at least for 3 hours, preferentially for 24 hours, more preferentially for 48 h, in order to enable said cells to aggregate into spheroids.

13. The method according to claim 12, further comprising a step of adding cells of a different type from the cells of each spheroid in order to provide a co-culture of two different type of cells.
